(19) United States
(12) Patent Application Publication
(10) Pub. No.: US 2008/0275089 A1
(43) Pub. Date: Nov. 6, 2008

(54) Compositions and methods to treat gastrointestinal disorders

(75) Inventor: John R. Ieni, Bloomfield, NJ (US)

Correspondence Address: LAHIVE & COCKFIELD, LLP;FLOOR 30, SUITE 3000, ONE POST OFFICE SQUARE, BOSTON, MA, 02109, US

(73) Assignee: EISAI CO., LTD., Tokyo (JP)

(21) Appl. No.: 11/901,642

(22) Filed: Sep. 18, 2007

Related U.S. Application Data
(63) Continuation of application No. 10/846,943, filed on May 17, 2004, now abandoned.
Application No. 10/846,943 is a continuation of application No. PCT/US02/36857, filed on Nov. 18, 2002.

(51) Int. Cl. A61K 31/435; A61P 1/04

(52) U.S. Cl. 514/338

(57) ABSTRACT

The invention provides safe and effective methods for treating and preventing dysphagia, lower esophageal mucosal rings, esophageal strictures, achalasia, gastric mucosal injuries, and bacterial infections. The methods comprise administering at least one proton pump inhibitor, optionally in combination with antibacterial compounds. In one embodiment, the proton pump inhibitor is rabeprazole, a pharmaceutically acceptable salt thereof and/or a stereoisomer thereof.

### RELATED APPLICATIONS

This application claims priority to U.S. patent application Ser. No. 10/846,943, filed May 16, 2004, which claims priority to International Application No. PCT/US02/36857 filed Nov. 18, 2002, U.S. Provisional Application No. 60/381,334 filed May 20, 2002, and to U.S. Provisional Application No. 60/331,585 filed Nov. 16, 2001, the disclosures of which are incorporated by reference herein in their entirety.

### FIELD OF THE INVENTION

The invention provides safe and effective methods for treating and preventing dysphagia, lower esophageal mucosal rings, esophageal strictures, achalasia, gastric mucosal injuries, and bacterial infections. The methods comprise administering at least one proton pump inhibitor. In one embodiment, the proton pump inhibitor is rabeprazole, a pharmaceutically acceptable salt thereof and/or a stereoisomer thereof.

### BACKGROUND OF THE INVENTION

Dysphagia is the inability to swallow or difficulty swallowing. Swallowing is a complicated action which is usually initiated voluntarily but always completed reflexively, whereby food is moved from the mouth through the pharynx and esophagus to the stomach. The act of swallowing occurs in three stages and requires the integrated action of the respiratory center and motor functions of multiple cranial nerves, and the coordination of the autonomic system within the esophagus. In the first stage, food is placed on the surface of the tongue. The tip of the tongue is placed against the hard palate. Elevation of the larynx and backward movement of the tongue forces the food through the isthmus of the fauces in the pharynx. In the second stage, the food passes through the pharynx. This involves constriction of the walls of the pharynx, backward bending of the epiglottis, and an upward and forward movement of the larynx and trachea. Food is kept from entering the nasal cavity by elevation of the soft palate and from entering the larynx by closure of the glottis and backward inclination of the epiglottis. During this stage, respiratory movements are inhibited by reflex. In the third stage, food moves down the esophagus and into the stomach. This movement is accomplished by momentum from the second stage, peristaltic contractions, and gravity. Although the main function of swallowing is the propulsion of food from the mouth into the stomach, swallowing also serves as a protective reflex for the upper respiratory tract by removing particles trapped in the nasopharynx and oropharynx, returning materials refluxed from the stomach into the pharynx, or removing particles propelled from the upper respiratory tract into the pharynx. The absence of an adequate swallowing reflex greatly increases the chance of pulmonary aspiration.

In the past, patients suffering from dysphagia had to undergo dietary changes or thermal stimulation treatment to regain adequate swallowing reflexes. Thermal stimulation involves immersing a mirror or probe in ice or a cold substance. The tonsillar fossa is stimulated with the mirror or probe and the patient closes his mouth and attempts to swallow. While these traditional methods are usually effective for treating dysphagia, they often require that the patient endure weeks or months of therapy.

There is a need in the art for new and improved treatments for dysphagia and other esophageal disorders. The invention is directed to these, as well as other, important ends.

### SUMMARY OF THE INVENTION

The invention provides methods for treating and/or preventing dysphagia in a patient by administering a therapeutically effective amount of at least one proton pump inhibitor. The methods can further comprise dilating the patient's esophagus; administering an endoscopic examination to the patient; and/or surgically incising, rupturing and/or excising the patient's lower esophageal mucosal rings or esophageal strictures.

The invention provides methods for treating and/or preventing lower esophageal mucosal rings or esophageal strictures in a patient by administering a therapeutically effective amount of at least one proton pump inhibitor. The methods can further comprise dilating the patient's esophagus; administering an endoscopic examination to the patient; and/or surgically incising, rupturing and/or excising the patient's lower esophageal mucosal rings or esophageal strictures.

The invention provides methods for reducing or eliminating a patient's need for dilation of lower esophageal mucosal rings or esophageal strictures by administering a therapeutically effective amount of at least one proton pump inhibitor.

The invention provides methods for reducing or eliminating a patient's need for surgically incising, rupturing and/or excising the patient's lower esophageal mucosal rings or esophageal strictures by administering a therapeutically effective amount of at least one proton pump inhibitor.

The invention provides methods for treating and/or preventing achalasia in a patient by administering a therapeutically effective amount of at least one proton pump inhibitor. The methods can further comprise dilating the patient's esophagus; and/or administering an endoscopic examination to the patient.

The invention provides methods for treating and/or preventing gastric mucosal injuries in a patient by administering a therapeutically effective amount of at least one proton pump inhibitor.

The invention provides methods for treating and/or preventing bacterial infections in a patient by administering a therapeutically effective amount of at least one proton pump inhibitor and, optionally, at least one antibacterial compound.

The invention provides methods for modulating bacterial growth in vitro and/or in vivo by administering an effective amount of at least one proton pump inhibitor, and, optionally, at least one antibacterial compound.

The invention is described in more detail below.

### DETAILED DESCRIPTION OF THE INVENTION

Esophageal dilation is a technique used to stretch and/or fracture a blocked portion(s) of the esophagus. Several types of esophageal dilators are available including, for example, simple dilators (e.g., bougies, mercury-filled bougies, tapered-tipped Maloney dilators, blunt-tipped Hurst dilators), guided wire bougies (e.g., polyvinyl bougies, Savary dilators), balloon dilators (e.g., passed over a guidewire or through an endoscope), and achalasia dilators. Simple dilators are a series of flexible dilators of increasing thickness. One or more of the dilators can be passed down through the patient's esophagus at a setting. It is a simple method of stretching and/or fracturing the blockage in the patient's esophagus. In a guided wire bougie, the physician can perform an endoscopy and place a flexible wire across the stricture. The scope can be removed and the wire can be left in place. At least one dilator with a hole through it from end to end can be guided down the esophagus and across the stricture. At the end of the procedure, the wire can be removed. In balloon dilation, a deflated balloon can be placed through the endoscope and across the stricture in the esophagus. When the balloon is inflated, it stretches and/or fractures the stricture. An achalasia dilator is similar to balloon dilation, but uses a larger balloon dilator, generally under x-ray control. In achalasia dilation, the spastic muscles in the lower esophagus are stretched and/or fractured with the balloon dilator.

A patient's esophagus can become blocked by, for example, esophageal strictures (e.g., peptic strictures), Schatzki's rings, ingestion of caustic agents, achalasia, tumors, or heredity. Esophageal strictures can be caused by acid reflux and/or a hiatal hernia which inflames and/or scars the esophagus. The fibrous scar contracts and narrows the esophageal opening. Schatzki's rings are narrow rings of benign fibrous tissue that constrict the lower esophagus. Schatzki's rings are also referred to as lower esophageal mucosal rings, and lower esophageal (Schatzki) rings. Achalasia is a persistent and marked spasm of the lower esophageal muscle which results in a persistent blockage of the esophagus.

“Patient” includes animals, preferably mammals, more preferably humans. “Patient” includes infants, children and adults, and includes males and females.

The invention provides methods for treating and/or preventing dysphagia in a patient in need thereof comprising administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt).

The invention provides methods for treating and/or preventing dysphagia in a patient in need thereof comprising dilating the patient's esophagus (e.g., lower esophagus), and subsequently administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt). The patient's esophagus can be dilated by any dilation procedure in the art, such as those described herein. In one embodiment, the patient's esophagus is dilated with a simple bougie. The dysphagia can be of any origin. In one embodiment, the dysphagia can be caused by lower esophageal mucosal rings (i.e., Schatzki's rings). In another embodiment, the dysphagia can be caused by esophageal strictures (e.g., peptic strictures, such as those caused by reflux esophagitis). In another embodiment, the dysphagia can be caused by achalasia. The methods can further comprise administering at least one proton pump inhibitor prior to dilating the patient's esophagus. The methods can further comprise administering an endoscopic examination to the patient prior to, during, and/or after dilating the patient's esophagus. The methods can further comprise surgically incising, rupturing and/or excising the patient's lower esophageal mucosal rings or esophageal strictures before and/or after dilating the patient's esophagus.

The invention provides methods for treating and/or preventing lower esophageal mucosal rings in a patient in need thereof comprising administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt).

The invention provides methods for treating and/or preventing lower esophageal mucosal rings in a patient in need thereof comprising dilating the patient's esophagus (e.g., lower esophagus), and subsequently administering a therapeutically effective amount of at least one proton pump inhibitor. The patient's esophagus can be dilated by any dilation procedure in the art, such as those described herein. In one embodiment, the patient's esophagus is dilated with a simple bougie. The methods can further comprise administering at least one proton pump inhibitor prior to dilating the patient's esophagus. The methods can further comprise administering an endoscopic examination to the patient prior to, during, and/or after dilating the patient's esophagus. The methods can further comprise surgically incising, rupturing and/or excising the patient's lower esophageal mucosal rings before and/or after dilating the patient's esophagus.

The invention provides methods for treating and/or preventing esophageal strictures in a patient in need thereof comprising administering a therapeutically effective amount of at least one proton pump inhibitor. The esophageal strictures can be peptic strictures, such as those caused by reflux esophagitis. In one embodiment, the proton pump inhibitor is rabeprazole, a stereoisomer thereof, and/or a pharmaceutically acceptable salt thereof (e.g., sodium salt).

The invention provides methods for treating and/or preventing esophageal strictures in a patient in need thereof comprising dilating the patient's esophagus, and subsequently administering a therapeutically effective amount of at least one proton pump inhibitor. The patient's esophagus can be dilated by any dilation procedure in the art, such as those described herein. In one embodiment, the patient's esophagus is dilated with a simple bougie. The methods can further comprise administering at least one proton pump inhibitor prior to dilating the patient's esophagus. The methods can further comprise administering an endoscopic examination to the patient prior to, during, and/or after dilating the patient's esophagus. The methods can further comprise surgically incising, rupturing and/or excising the patient's esophageal strictures before and/or after dilating the patient's esophagus.

The invention provides methods for treating and/or preventing dysphagia in a patient in need thereof comprising surgically incising, rupturing and/or excising the patient's lower esophageal mucosal rings, and subsequently administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt). The methods can further comprise administering at least one proton pump inhibitor prior to surgically incising, rupturing and/or excising the patient's lower esophageal mucosal rings. The methods can further comprise administering an endoscopic examination to the patient prior to, during, and/or after surgically incising, rupturing and/or excising the patient's lower esophageal mucosal rings. The methods can further comprise dilating the patient's esophagus (e.g., lower esophagus) prior to and/or after surgically incising, rupturing and/or excising the patient's lower esophageal mucosal rings.

The invention provides methods for treating and/or preventing dysphagia in a patient in need thereof comprising surgically incising, rupturing and/or excising the patient's esophageal strictures, and subsequently administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt). The methods can further comprise administering at least one proton pump inhibitor prior to surgically incising, rupturing and/or excising the patient's esophageal strictures. The methods can further comprise administering an endoscopic examination to the patient prior to, during, and/or after surgically incising, rupturing and/or excising the patient's esophageal strictures. The methods can further comprise dilating the patient's esophagus prior to and/or after surgically incising, rupturing and/or excising the patient's esophageal strictures.

The invention provides methods for treating and/or preventing lower esophageal mucosal rings in a patient in need thereof comprising surgically incising, rupturing and/or excising the patient's lower esophageal mucosal rings, and subsequently administering a therapeutically effective amount of at least one proton pump inhibitor.

In one embodiment, the proton pump inhibitor is rabeprazole, a stereoisomer thereof, and/or a pharmaceutically acceptable salt thereof (e.g., sodium salt). The methods can further comprise administering at least one proton pump inhibitor prior to surgically incising, rupturing and/or excising the patient's lower esophageal mucosal rings. The methods can further comprise administering an endoscopic examination to the patient prior to, during, and/or after surgically incising, rupturing and/or excising the patient's lower esophageal mucosal rings. The methods can further comprise dilating the patient's esophagus (e.g., lower esophagus) prior to and/or after surgically incising, rupturing and/or excising the patient's lower esophageal mucosal rings.

The invention provides methods for treating and/or preventing esophageal strictures in a patient in need thereof comprising surgically incising, rupturing and/or excising the patient's esophageal strictures, and subsequently administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt). The methods can further comprise administering at least one proton pump inhibitor prior to surgically incising, rupturing and/or excising the patient's esophageal strictures. The methods can further comprise administering an endoscopic examination to the patient prior to, during, and/or after surgically incising, rupturing and/or excising the patient's esophageal strictures. The methods can further comprising dilating the patient's esophagus prior to and/or after surgically incising, rupturing and/or excising the patient's esophageal strictures.

The invention provides methods for reducing or eliminating a patient's need for dilation of lower esophageal mucosal rings or esophageal strictures comprising administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt). The methods can further comprise dilating the lower esophageal mucosal rings or esophageal strictures before and/or after administering the therapeutically effective amount of at least one proton pump inhibitor. The methods can further comprise administering an endoscopic examination to the patient before, during, and/or after administering a therapeutically effective amount of at least one proton pump inhibitor.

The invention provides methods for reducing or eliminating a patient's need for surgically incising, rupturing and/or excising the lower esophageal mucosal rings or esophageal strictures comprising administering a therapeutically effective amount of at least one proton pump inhibitor. The methods can further comprising dilating and/or surgically incising, rupturing and/or excising the lower esophageal mucosal rings or esophageal strictures before and/or after administering the therapeutically effective amount of at least one proton pump inhibitor. The methods can further comprise administering an endoscopic examination to the patient before, during, and/or after administering a therapeutically effective amount of at least one proton pump inhibitor.

The term “eliminating” means that the methods of the invention prevent the need for any future dilation and/or surgical treatment due to lower esophageal mucosal rings or esophageal strictures. The term “reducing” means that the methods of the invention allow greater periods of time elapse between dilations and/or surgical treatments when compared to the amount of time that would elapse between dilations and/or surgical treatments without the methods of the invention.

The invention provides methods for treating and/or preventing achalasia in a patient in need thereof comprising administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt).

The invention provides methods for treating and/or preventing achalasia in a patient in need thereof comprising dilating the patient's esophagus, and subsequently administering a therapeutically effective amount of at least one proton pump inhibitor. In one embodiment, the proton pump inhibitor is rabeprazole, a stereoisomer thereof, and/or a pharmaceutically acceptable salt thereof (e.g., sodium salt). The patient's esophagus can be dilated by any dilation procedure in the art, such as those described herein. The methods can further comprise administering at least one proton pump inhibitor prior to dilating the patient's esophagus. The methods can further comprises administering an endoscopic examination to the patient prior to, during, and/or after dilating the patient's esophagus.

In another embodiment, the invention provides methods for preventing and/or treating a gastric mucosal injury by administering a therapeutically effective amount of at least one proton pump inhibitor to a patient in need thereof. The gastric mucosal injury can be caused by, for example, alcohol (e.g., ethanol) and/or drugs (e.g., prescription drugs and/or street drugs). For example, the invention provides methods for treating ethanol-induced gastric mucosal injury by administering a therapeutically effective amount of at least one proton pump inhibitor to a patient in need thereof. In one embodiment, the proton pump inhibitor is rabeprazole, a stereoisomer thereof, and/or a pharmaceutically acceptable salt thereof (e.g., sodium salt).

In other embodiments, the invention provides compositions comprising at least one proton pump inhibitor and at least one antibacterial compound, and, optionally, at least one other proton pump inhibitor. The compositions comprise a pharmaceutically acceptable carrier. The invention provides pharmaceutical kits comprising at least one proton pump inhibitor and at least one antibacterial compound, and, optionally, at least one other proton pump inhibitor. In the kits of the invention, the at least one proton pump inhibitor, the at least one antibacterial compound, and, optionally, the at least one other proton pump inhibitor, are separate components in the kit or are in the form of a composition in the kit. In one embodiment, the proton pump inhibitor is rabeprazole, a stereoisomer thereof, and/or a pharmaceutically acceptable salt thereof (e.g., sodium salt).

In other embodiments, the invention provides methods for preventing and/or treating bacterial infections by administering to a patient in need thereof a therapeutically effective amount of at least one proton pump inhibitor. In another embodiment, the invention provides methods for preventing and/or treating bacterial infections by administering to a patient in need thereof a therapeutically effective amount of at least one proton pump inhibitor and at least one antibacterial compound. In other embodiments, the invention provides methods for preventing and/or treating bacterial infections by administering to a patient in need thereof a therapeutically effective amount of at least one proton pump inhibitor (e.g., rabeprazole, a stereoisomer thereof and/or a pharmaceutically acceptable salt thereof), at least one other proton pump inhibitor, and, optionally, at least one antibacterial compound. The proton pump inhibitor, proton pump inhibitor and/or the antibacterial compound can be administered to the patient separately or in the form of a composition. In one embodiment, the proton pump inhibitor is rabeprazole, a stereoisomer thereof, and/or a pharmaceutically acceptable salt thereof (e.g., sodium salt).

In other embodiments, the invention provides methods for modulating bacterial growth in vivo and/or in vitro by administering an effective amount of at least one proton pump inhibitor. In another embodiment, the invention provides methods for modulating bacterial growth in vivo and/or in vitro by administering an effective amount of at least one proton pump inhibitor and at least one antibacterial compound. In other embodiments, the invention provides methods for modulating bacterial growth in vivo and/or in vitro by administering an effective amount of at least one proton pump inhibitor (e.g., rabeprazole, a stereoisomer thereof and/or a pharmaceutically acceptable salt thereof), at least one other proton pump inhibitor, and at least one antibacterial compound. The proton pump inhibitor, the antibacterial compound and/or the proton pump inhibitor can be administered separately or in the form of a composition. In one embodiment, the proton pump inhibitor is rabeprazole, a stereoisomer thereof, and/or a pharmaceutically acceptable salt thereof (e.g., sodium salt).

“Modulating bacterial growth” includes inhibiting the growth of bacteria; reducing the rate at which the bacteria grows (i.e., compared to the rate at which untreated bacteria grows); and/or killing the bacteria. The growth of bacteria can be modulated in vitro and/or in vivo.

It has been unexpectedly discovered that the combined use of a proton pump inhibitor and at least one antibacterial compound enhances the effect of the antibacterial compound. The enhanced effect can be achieved by administering the compounds separately or in the form of a composition. The effect can be further enhanced by administering at least one other proton pump inhibitor.

When administered separately, the proton pump inhibitors and antibacterial compounds can be administered about the same time as part of an overall treatment regimen, i.e., as a combination therapy. “About the same time” includes administering the proton pump inhibitors and at least one antibacterial compound at the same time, at different times on the same day, or on different days, as long as they are administered as part of an overall treatment regimen.

“Antibacterial compounds” include any antibacterial compounds and antibiotics in the art, including derivatives and metabolites thereof. Exemplary antibacterial compounds include beta-lactam compounds, quinolone compounds, cephalosporin compounds, carbapenem compounds, glycopeptide antibiotics, lipopeptide antibiotics, monobactam compounds, aminoglycoside antibiotics, streptogramin compounds, oxazolidinone compounds, macrolide compounds, azalide compounds, ketolide compounds, tetracycline compounds, lincosamide compounds, penicillin compounds, beta-lactamase inhibitors, efflux pump inhibitors, and the like. “Antibacterial compounds” include any other antibiotic, derivative thereof or metabolite thereof that does not fit into the categories described herein. The antibacterial compounds can be naturally produced and/or synthetically produced.

Any quinolone compound in the art can be used in the compositions and methods of the invention. “Quinolone compounds” includes fluoroquinolone compounds. Exemplary quinolone compounds include nalidixic acid, cinoxacin, oxolinic acid, norfloxacin, lomefloxacin, enoxacin, ofloxacin, ciprofloxacin, levofloxacin, sparfloxacin, gatifloxacin, grepafloxacin, gemifloxacin, sitafloxacin, moxifloxacin, trovafloxacin, alatrofloxacin, clinafloxacin, DC-756, Y-34867, T-3811, WQ-3034, S-34109, HSR-903, CFC-222, and the like.

Any cephalosporin compound in the art can be used in the compositions and methods of the invention. “Cephalosporin compounds” include cephem antibiotics and oxacephem antibiotics. Exemplary cephalosporin compounds include E1077, E1101, S-1090, FK041, MC-02,479, ME1209, Ro 63-9141, cefadroxil, cephalexin, cephradine, cephalothin, cephapirin, cefazolin, cefaclor, cefuroxime, cefprozil, loracarbef, cefamandole, cefoxitin, cefinetazole, cefotetan, cefonicid, cefixime, cefpodoxime, cefibuten, cefoperazone, cefotaxime, ceftizoxime, ceftazidime, cefdinir, ceftriaxone, cefepime, cefditoren pivoxil and the like. In one embodiment, the cephalosporin compound is E1077 or E1101.

Any carbapenem compound in the art can be used in the compositions and methods of the invention. Carbapenem compounds include penem compounds (e.g., MEN 10700). Exemplary carbapenem compounds include E1010, J-1 11,225, J-111,347, J-114,871, L-786,392, MK-826, S-4661, biapenem, sanfetrinem, imipenem, meropenem, L-084, LJC 11,036, KR-21056, KR-21012, CS-834, R-95867, DZ-2640, DU-6681, GV 104326, MEN 10700, ritipenem, and the like. In one embodiment, the carbapenem compound is E1010.

Any glycopeptide and lipopeptide antibiotic in the art can be used in the compositions and methods of the invention. Exemplary glycopeptide and lipopeptide antibiotics include vancomycin, teicoplanin, BI 397, daptomycin, LY312607, LY314015, LY333328, and the like.

Any monobactam compound in the art can be used in the compositions and methods of the invention. Exemplary monobactam compounds include aztreonam.

Any aminoglycoside antibiotics in the art can be used in the compositions and methods of the invention. Exemplary aminoglycoside antibiotics include sisomicin, streptomycin, micronomicin, gentamicin, tobramycin, netilmicin, amikacin, kanamycin and the like.

Any streptogramin compound in the art can be used in the compositions and methods of the invention. Exemplary streptogramin compounds include virginiamycin and the like.

Any oxazolidinone compound in the art can be used in the compositions and methods of the invention. Exemplary oxazolidinone compounds include linezolid, PNU-107922, PNU-140457, PNU-172576, PNU-176798 and the like.

Any macrolide, azalide or ketolide compound in the art can be used in the compositions and methods of the invention. Exemplary macrolide, azalide and ketolide compounds include erythromycin, clarithromycin, troleandomycin, roxithromycin, dirithromycin, azithromycin, A-181785, A-184656, A-241550, CP-279,107, CP-544,372, HMR 3004-HMR 3647, TE-802, TE-810, telithromycin, erythromycin/sulfisoxazole and the like.

Any tetracycline compound in the art can be used in the compositions and methods of the invention. Glycylcycline compounds fall within the scope of tetracyclicine compounds. Exemplary tetracycline compounds include tetracycline, oxytetracycline, demeclocycline, methacycline, doxycycline, minocycline, terbutyl-minocycline, and the like.

Any lincosamide antibiotic in the art can be used in the compositions and methods of the invention. Exemplary lincosamide antibiotics include lincomycin, clindamycin, pirlimycin and the like.

Any penicillin compound in the art can be used in the compositions and methods of the invention. Exemplary penicillin compounds include penicillin G, penicillin V, ampicillin, ampicillin/sulbactam, amoxicillin, amoxicillin/clavulanate, hetacillin, methicillin, cloxacillin, dicloxacillin, nafcillin, oxacillin, azlocillin, carbenicillin, mezlocillin, piperacillin, piperacillin/tazobactam, ticarcillin, ticarcillin/clavulanate, and the like.

Any beta-lactamase inhibitor in the art can be used in the compositions and methods of the invention. Exemplary beta-lactamase inhibitors include aztreonzm, cefotetan, loracarbef, cefoxitin, meropenem, imipenem/cilastatin, cefinase, clavulanate, sulbactam, tazobactam, and the like.

Any efflux pump inhibitor in the art can be used in the compositions and methods of the invention. Exemplary efflux pump inhibitors include CCCP, PSC-833, MC-04,124, MC-510027, MC-207110, MC-02595, and the like.

Any other antibiotic in the art not falling into one of the above categories can be used in the compositions and methods of the invention. Such other antibiotics can include trimethoprim-sulfamethoxazole, chloramphenicol, metronidazole, mupirocin, everninomicin, rifamycin, clindamycin, colistimethate, quinupristin/dalfopristin, vancomycin, and the like.

The invention also provides methods for potentiating the at least one proton pump inhibitor by further administering at least one beta-lactamase inhibitor. The at least one proton pump inhibitor and the at least one beta-lactamase inhibitor can be administered separately or in the form of a composition. The invention also provides methods for potentiating the combination of at least one proton pump inhibitor and at least one antibacterial compound (i.e., other than a beta-lactamase inhibitor) by further administering at least one beta-lactamase inhibitor. The at least one proton pump inhibitor, the at least one antibacterial compound, and the at least one beta-lactamase inhibitor can be administered separately or in the form of a composition. Any beta-lactamase inhibitor in the art can be used. Exemplary beta-lactamase inhibitors include aztreonzm, cefotetan, loracarbef, cefoxitin, meropenem, imipenem/cilastatin, cefinase, clavulanate, sulbactam, tazobactam, and the like. In one embodiment, the invention provides methods for administering a therapeutically effective amount of at least one proton pump inhibitor (e.g., rabeprazole, a stereoisomer thereof, and/or a pharmaceutically acceptable salt thereof), at least one antibacterial compound (i.e., other than a beta-lactamase inhibitor), and at least one beta-lactamase inhibitor to treat a bacterial infection in a patient in need thereof, or to modulate the growth of bacteria. The combinations can include, for example, rabeprazole, a stereoisomer thereof, and/or a pharmaceutically acceptable salt thereof, amoxicillin and clavulanate; rabeprazole, a stereoisomer thereof, and/or a pharmaceutically acceptable salt thereof, piperacillin and clavulanate; rabeprazole, a stereoisomer thereof, and/or a pharmaceutically acceptable salt thereof, ticarcillin and tazobactam; or rabeprazole, a stereoisomer thereof, and/or a pharmaceutically acceptable salt thereof, ampicillin and sulbactam. One skilled in the art will recognize that other combinations can be used.

The invention provides methods for potentiating the at least one proton pump inhibitor by further administering at least one efflux pump inhibitor. The at least one proton pump inhibitor and the at least one efflux pump inhibitor can be administered separately or in the form of a composition. The invention also provides methods for further potentiating the combination of at least one proton pump inhibitor and at least one antibacterial compound (i.e., other than an efflux pump inhibitor) by further administering at least one efflux pump inhibitor. The at least one proton pump inhibitor, the at least one antibacterial compound and the at least one efflux pump inhibitor can be administered separately or in the form of a composition. The efflux pump inhibitor can be any in the art. Exemplary efflux pump inhibitors include CCCP, PSC-833, MC-04,124, MC-510027, MC-207110, MC-02595, and the like. In one embodiment, the methods can comprise administering a therapeutically effective amount of at least one proton pump inhibitor (e.g., rabeprazole, a stereoisomer thereof, and/or a pharmaceutically acceptable salt thereof), at least one antibacterial compound (e.g., a quinolone, such as levofloxicin), and at least one efflux pump inhibitor to treat a bacterial infection (e.g., P. aeruginosa) in a patient in need thereof, or to modulate bacterial growth (e.g., P. aeruginosa) in vivo or in vitro. In other embodiments, the methods can comprise administering a therapeutically effective amount of at least one proton pump inhibitor (e.g., rabeprazole, a stereoisomer thereof, and/or a pharmaceutically acceptable salt thereof), at least one antibacterial compound (e.g., a macrolide, such as azithromycin, clarithromycin and/or erythromycin), and at least one efflux pump inhibitor to treat a bacterial infection (e.g., E. coli, H. influenzae, K. pneumoniae) in a patient in need thereof, or to modulate bacterial growth (e.g., E. coli, H. influenzae, K. pneumoniae) in vivo or in vitro.

Bacterial infections that can be treated with the methods of the invention include any in the art including, for example, urinary tract infections, respiratory tract infections, skin infections, urethral infections, sexually transmitted diseases (e.g., gonococcal infections, chlamydial infections), bone infections, joint infections, infectious diarrhea, typhoid fever, prostatitis, sinusitis, chronic bronchitis, pneumonia, gastrointestinal infections (e.g., H. pylori), intra-abdominal infections, gynecologic and pelvic infections, anthrax, and the like.

Bacterial infections that can be treated in a patient can be infections caused by any bacteria in the art including, for example, Helicobacter species (e.g., pylori), Clostridium species (e.g., difficile, botulinum), Mycobacterium species, Staphylococcus species (e.g., aureus), Pseudomonas species (e.g., aeruginosa), Streptoccus species (e.g., pneumoniae, pyogenes), Enterobacteriaceae species, Enterococcus species, Mycoplasma species (e.g., pneumoniae), Chlamydia species (e.g., pneumoniae, trachomatis), Bacteroides species, Bacillus species (e.g., anthracis), Enterobacter species, Klebsiella species (e.g., pneumoniae), Haemophilus species (e.g., influenzae, parainfluenzae), Moraxella species (e.g., catarrhalis), Proteus species (e.g., mirabilis), Acinetobacter species, Serratia species, E. coli, and the like. In one embodiment, the methods are directed to preventing and/or treating infections caused by Staphylococcus aureus. In another embodiment, the methods are directed to preventing and/or treating infections caused by Pseudomonas aeruginosa. In other embodiment, the methods are directed to preventing and/or treating infections caused by Helicobacter pylori. In other embodiments, the methods are directed to preventing and/or treating infections caused by Clostridium difficile.

Bacteria that can be modulated in vivo and/or in vitro by the methods of the invention can be any in the art including, for example, Helicobacter species (e.g., pylori), Clostridium species (e.g., difficile, botulinum), Mycobacterium species, Staphylococcus species (e.g., aureus), Pseudomonas species (e.g., aeruginosa), Streptoccus species (e.g., pneumoniae, pyogenes), Enterobacteriaceae species, Enterococcus species, Mycoplasma species (e.g., pneumoniae), Chlamydia species (e.g., pneumoniae, trachomatis), Bacteroides species, Bacillus species (e.g., anthracis), Enterobacter species, Klebsiella species (e.g., pneumoniae), Haemophilus species (e.g., influenzae, parainfluenzae), Moraxella species (e.g., catarrhalis), Proteus species (e.g., mirabilis), Acinetobacter species, Serratia species, E. coli, and the like. In one embodiment, the method is directed to modulating the growth of Helicobacter pylori. In one embodiment, the method is directed to modulating the growth of Staphylococcus aureus. In another embodiment, the method is directed to modulating the growth of Pseudomonas aeruginosa. In another embodiment, the method is directed to modulating the growth of Clostridium difficile.

The invention also provides methods for treating and/or preventing H. pylori in order to prevent and/or treat cancer by administering a therapeutically effective amount of at least one proton pump inhibitor and, optionally, at least one antibacterial compound. The proton pump inhibitor is preferably rabeprazole, a stereoisomer thereof, and/or a pharmaceutically acceptable salt thereof. In other embodiments, at least one proton pump inhibitor (e.g., rabeprazole, a stereoisomer thereof, and/or a pharmaceutically acceptable salt thereof) is administered in conjunction (i.e., separately or in the form of a composition) with at least one proton pump inhibitor other than rabeprazole, and, optionally, at least one antibacterial compound. The cancer can be any cancer in the art, but is generally a gastrointestinal cancer, such as gastric cancer, duodenal cancer, esophageal cancer, laryngeal cancer, and the like.

The invention provides methods for treating and/or preventing H. pylori in order to prevent and/or treat ulcers (e.g., esophageal ulcers, duodenal ulcers, stomach ulcers, jejenum ulcers, ileum ulcers) by administering a therapeutically effective amount of at least one proton pump inhibitor and, optionally, at least one antibacterial compound. The proton pump inhibitor is preferably rabeprazole, a stereoisomer thereof, and/or a pharmaceutically acceptable salt thereof. In other embodiments, at least one proton pump inhibitor (e.g., rabeprazole, a stereoisomer thereof, and/or a pharmaceutically acceptable salt thereof) is administered in conjunction (i.e., separately or in the form of a composition) with at least one proton pump inhibitor other than rabeprazole, and, optionally, at least one antibacterial compound.

The invention provides methods for treating and/or preventing H. pylori in order to prevent and/or treat dyspepsia by administering a therapeutically effective amount of at least one proton pump inhibitor and, optionally, at least one antibacterial compound. The proton pump inhibitor is preferably rabeprazole, a stereoisomer thereof, and/or a pharmaceutically acceptable salt thereof. In other embodiments, at least one proton pump inhibitor (e.g., rabeprazole, a stereoisomer thereof, and/or a pharmaceutically acceptable salt thereof) is administered in conjunction (i.e., separately or in the form of a composition) with at least one proton pump inhibitor other than rabeprazole, and, optionally, at least one antibacterial compound.

Any proton pump inhibitor in the art can be used in the compositions and methods described herein. Exemplary proton pump inhibitors include rabeprazole, omeprazole, lansoprazole, esomeprazole, pantoprazole and the like.

In one embodiment, the proton pump inhibitor is a pyridine derivative of formula (I), pharmaceutically acceptable salts thereof, and/or stereoisomers thereof:

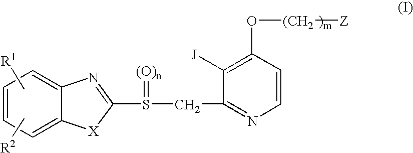

wherein R¹ and R² are each independently a hydrogen atom, a halogen atom, a lower alkyl, lower alkoxy, halogenated lower alkyl, lower alkoxycarbonyl or carboxyl group;

X is —O—, —S— or ═N—R³, wherein R³ is a hydrogen atom or a lower alkyl, phenyl, benzyl or lower alkoxycarbonyl group; and

Z is:

—O(CH₂)ₚ—O—R⁴ 1.

wherein p is an integer of 1 to 3 and R⁴ is a hydrogen atom or a lower alkyl, aryl or aralkyl group,

—O—(CH₂)_{q}—R⁴ 2.

wherein q is an integer of 1 to 3 and R⁴ is a halogen atom or an alkoxycarbonyl, aryl or heteroaryl group,

—O—(CH₂)ᵣO—(CH₂)ₛ—O—R⁶ 3.

wherein r and s are each independently an integer of 1 to 5 and R⁶ is a hydrogen atom or a lower alkyl group,

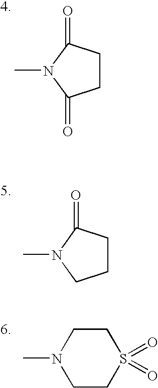

—S(O)ₜ-A 7.
wherein t is an integer of 0 to 2, and A is a lower alkyl, alkoxycarbonylmethyl, pyridyl, furyl,

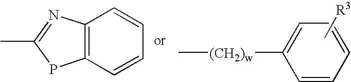

wherein P is —NH—, —O— or —S—, and w is an integer of 0 or 1;

—N(R⁸)CH₂—C₆H₅ 8.
wherein R⁸ is an acetoxy or lower alkyl group;

—OR⁹ 9.

wherein R⁹ is a hydrogen atom, a lower alkyl or aryl group;

n is an integer of 0 to 2; m is an integer of 2 to 10, and J and K are each independently a hydrogen atom or a lower alkyl group, with the proviso that when Z is a group falling under the above category (9), then R⁹ is a lower alkyl group and m stands for an integer of 3 to 10, and pharmaceutically acceptable salts thereof.

The same definitions for R¹, R², X, n, J, K, Z and m are used throughout the specification that follows and in the appended claims.

Also disclosed are pharmaceutical compositions containing one or more of these compounds as the active ingredient(s) in a pharmaceutically acceptable carrier, adjuvant or vehicle.

In the definition of the compounds of formula (I), the lower alkyl group defined with respect to R¹, R², R³, R⁴, R⁶, R⁷, R⁸, A, J and K can be a straight-chain or branched alkyl group having 1 to 6 carbon atoms. Examples include methyl, ethyl, n-propyl, n-butyl, isopropyl, isobutyl, 1-methylpropyl, tert-butyl, n-pentyl, 1-ethylpropyl, isoamyl and n-hexyl groups, among which methyl and ethyl groups are most preferred.

The lower alkoxy group and the lower alkoxy moiety of the lower alkoxycarbonyl group defined above with respect to R¹ and R² can be an alkoxy group derived from the above lower alkyl group. Methoxy and ethoxy groups are most preferred.

The halogen atom defined above includes chlorine, bromine, iodine or fluorine.

The aryl group defined above with respect to R⁴ and R⁵ can be phenyl, tolyl, xylyl, napthyl or the like, which can be substituted with a lower alkoxy or hydroxyl group, a halogen atom or the like.

Examples of the arylalkyl defined above with respect to R⁴ include benzyl and phenethyl groups.

Examples of the heteroaryl group defined above with respect to R⁵ include pyridyl and furyl groups.

In the definition of Z in formula (I), groups 1, 2, 3, 4, 5 and 9 are preferred; and group 9 is the most preferred. As for R¹ and R², hydrogens for both and then a combination of a lower alkyl (e.g., methyl) for R¹ and hydrogen for R² are preferred. X is preferably ═NR³, where R³ is hydrogen. A preferred value for n is 1. The preferred substituents for J and K are both hydrogen or where J is lower alkyl (e.g., methyl), and K is hydrogen, or when J is hydrogen and K is lower alkyl (e.g., methyl). Thus, J or K are independently preferably hydrogen or methyl, most preferably J is methyl and K is hydrogen.

In another embodiment, the compounds of formula (I) are compounds of formula (A), pharmaceutically acceptable salts thereof, and/or stereoisomers thereof:

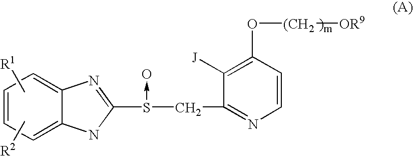

wherein R¹, R², J, m and R⁹ have the same meanings as defined above.

In formula (A), the preferred R¹ and R² substituents are both hydrogen, or R¹ is 5-lower alkoxy, 5-lower alkyl or 5-halogenated lower alkyl and R² is hydrogen. The preferred substituent for J is hydrogen or methyl; the preferred value for m is in the range of 3 to 10, the most preferred being 3; and the preferred R⁹ substituent is lower alkyl (e.g., methyl), or aryl. Among these possibilities for the compounds of formula (A), the preferred combination is when R¹ and R² are both hydrogen, J is methyl, m is 3 and R⁹ is methyl.

Another group of preferred compounds in formula (A) are combinations of the above substituents where both R¹ and R² are hydrogen, J is hydrogen, m is 3 and R⁹ is methyl.

Another group of preferred compounds falling within formula (A) is when both R¹ and R² are hydrogen, J is methyl, m is 2 and R⁹ is benzyl.

In another embodiment, the compounds of formula (I) are compounds of formula (B), pharmaceutically acceptable salts thereof, and/or stereoisomers thereof:

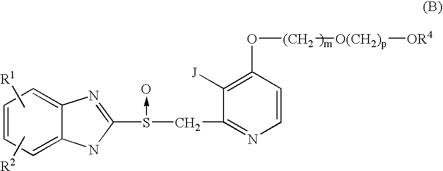

wherein R¹, R², J, p, m and R⁴ have the same meanings as given above.

In formula (B), the preferred substituents for R¹ and R² are both hydrogen; or when R¹ is 5-lower alkoxy, 5-lower alkyl or 5-halogenated lower alkyl, R² is hydrogen. The preferred value of m is 2 or 3; the preferred value for p is 2 or 3; and the preferred substituent for R⁴ is methyl or benzyl. Of the above possibilities for formula (B), the most preferred combination is where R¹ is 5-methyl, R² is hydrogen, J is methyl, m is 2, p is 2 and R⁴ is methyl.

In another embodiment, the compound of formula I is a compound of formula (C), a pharmaceutically acceptable salt thereof, and/or a stereoisomer thereof:

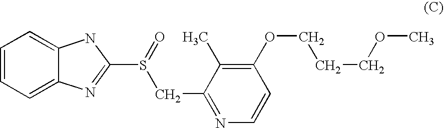

Preferably, the compound of formula (C) is a sodium salt, which is known as rabeprazole sodium or ACIPHEX® (Eisai Inc., Teaneck, N.J.).

Although the compounds of the invention can be present as a hydrate or as a stereoisomer, the hydrates and stereoisomers are included within the scope of the invention. For example, the compound of formula (C) can be s compound of formula (D) or a pharmaceutically acceptable salt thereof (e.g., a sodium salt):

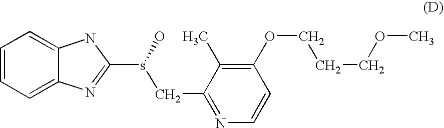

The compound of formula (D) is R(+) rabeprazole.

Alternatively, the compound of formula (C) can be a compound of formula (E) or a pharmaceutically acceptable salt thereof (e.g., a sodium salt):

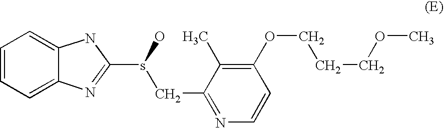

The compound of formula (E) is S(−) rabeprazole.

The compounds of the invention can be administered as any pharmaceutically acceptable salt in the art. Pharmaceutically acceptable salts are known in the art and include those of inorganic acids, such as hydrochloride, sulfate, hydrobromide, sulfate, and phosphate; those of organic acids, such as formate, acetate, maleate, tartrate, trifluoroacetate, methanesulfonate, benzenesulfonate and toluenesulfonate, and those of amino acids such as arginine, aspartic acid and glutamic acid. When certain substituents are selected, the compounds of the invention can form, for example, alkali metal salts, such as sodium or potassium salts; alkaline earth metal salts, such as calcium or magnesium salts; organic amine salts, such as a salt with trimethylamine, triethylamine, pyridine, picoline, dicyclohexylamine or N,N′-dibenzylethylenediamine. One skilled in the art will recognize that the compounds of the invention can be made in the form of any of these or of any other pharmaceutically acceptable salt. For example, compounds represented by formula (I), wherein X is ═N—R³ and R³ is a hydrogen atom, or compounds represented by formula (I), wherein Z is a group falling under the category 7 and B is a group of —NH—, can be present as a metal salt, such as sodium, potassium, magnesium or calcium.

The pyridine derivatives and proton pump inhibitors are commercially available and/or can be prepared by processes known in the art and described, for example, in U.S. Pat. No. 5,045,552, the disclosure of which is incorporated by reference herein in its entirety. Rabeprazole sodium is commercially available as ACIPHEX® from Eisai Inc., Teaneck, N.J. Methods for preparing R(+) rabeprazole are described in WO 99/55157, the disclosure of which is incorporated by reference herein in its entirety. Methods for preparing S(−) rabeprazole are described in WO 99/55158, the disclosure of which is incorporated by reference herein in its entirety.

A therapeutically effective dosage regimen for treating the diseases described herein with the proton pump inhibitors and/or antibacterial compounds is selected in accordance with a variety of factors, including the age, weight, sex, and medical condition of the patient, the severity of the disease, the route of administration, pharmacological considerations such as the activity, efficacy, pharmacokinetic and toxicology profiles of the particular proton pump inhibitor and/or antibacterial compound, whether a drug delivery system is used and whether the proton pump inhibitor and/or antibacterial compound is administered as part of a drug combination.

The proton pump inhibitors can be administered in amounts of about 0.01 to about 200 mg per day, preferably about 0.05 to about 50 mg per day, more preferably about 0.1 to about 40 mg per day, still more preferably about 10 to about 30 mg per day, most preferably about 20 mg per day. The compounds and/or compositions can be administered once a day or in divided doses, for example from 2 to 4 times a day, preferably once per day. One skilled in the art will recognize that when the compounds and/or compositions of the invention are administered to infants or children, the dose can be smaller than the dose administered to adults, and that the dose can be dependent upon the size and weight of the patient.

In preferred embodiments of the methods described herein, rabeprazole sodium, which is commercially available as ACIPHEX® (Eisai Inc., Teaneck, N.J.), is administered as a delayed-release, enteric-coated tablet containing 20 milligrams rabeprazole sodium. The tablets can be administered one to about four times a day. In preferred embodiments, one 20 milligram ACIPHEX® tablet is administered once a day for the methods described herein. One skilled in the art will appreciate that when rabeprazole sodium is administered to infants or children, the dose can be smaller than the dose that is administered to adults.

The antibacterial compounds can be prepared by processes known in the art or can be obtained from commercial sources, and can be administered in therapeutically effective doses that are known in the art, such as those described in The Physician's Desk Reference.

The proton pump inhibitors and/or antibacterial compounds can be administered orally, topically, parenterally, by inhalation (nasal or oral), or rectally in dosage unit formulations containing conventional nontoxic pharmaceutically acceptable carriers, adjuvants, and vehicles as desired. The term parenteral as used herein includes subcutaneous, intravenous, intramuscular, intrasternal injection, or infusion techniques. Preferably, the proton pump inhibitors are orally administered as tablets.

Injectable preparations, for example, sterile injectable aqueous or oleaginous suspensions can be formulated according to the known art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), preservatives and/or stabilizers. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids such as oleic acid find use in the preparation of injectables. The preparations can be lyophilized by methods known in the art.

Solid dosage forms for oral administration can include capsules, tablets, sublingual tablets, powders, granules and gels; most preferably tablets. The solid dosage form can be a solid microencapsulated dosage, such as a microencapsulated powder, microencapsulated granules or a microencapsulated gel. A solid dosage form for oral administration can be prepared by mixing an active principle with filler and, if necessary, binder, disintegrating agent, lubricant, coloring agent, corrigent or the like and converting the obtained mixture into a tablet, coated tablet, granule, powder or capsule. Examples of the filler include lactose, corn starch, sucrose, glucose, sorbitol, crystalline cellulose and silicon dioxide, while those of the binder include polyvinyl alcohol, polyvinyl ether, ethylcellulose, methylcellulose, acacia, tragacanth, gelatin, shellac, hydroxypropylcellulose, hydroxypropylstarch and polyvinylpyrrolidone. Examples of the disintegrating agent include starch, agar, gelatin powder, crystalline cellulose, calcium carbonate, sodium hydrogencarbonate, calcium citrate, dextrin and pectin, while those of the lubricant include magnesium stearate, talc, polyethylene glycol, silica and hardened vegetable oils. The coloring agent can be any one which is permitted to be added to drugs. Examples of the corrigent include cacao powder, mentha herb, aromatic powder, mentha oil, borneol and powdered cinnamon bark. The tablets and granules can be, if necessary, coated with sugar, gelatin or the like. Preferably, the tablets have an enteric coating.

In other embodiments, the solid dosage form can be packaged as granules or a powder in a pharmaceutically acceptable carrier, where the granules or powder are removed from the packaging and sprinkled on food or mixed with a liquid, such as water or juice. In this embodiment, the active compound can be mixed with flavoring or sweetening agents. The packaging material can be plastic, polyester films, nylon films, polyolefin films, shrink packing films, coated paper, or any material that prevents water or moisture from reaching the granules and/or powder.

Liquid dosage forms for oral administration can include pharmaceutically acceptable emulsions, solutions, suspensions, and syrups containing inert diluents commonly used in the art, such as water. The liquid dosage form can be a microencapsulated liquid, including microencapsulated emulsions, microencapsulated solutions, microencapsulated suspensions and microencapsulated syrups. Such compositions can also comprise adjuvants, such as wetting agents, emulsifying and suspending agents, and sweetening, flavoring, and perfuming agents.

For administration by oral or nasal inhalation, the compounds and compositions can be delivered from an insufflator, a nebulizer or a pressured pack or other convenient mode of delivering an aerosol spray. Pressurized packs can include a suitable propellant. Alternatively, for administration by oral or nasal inhalation, the compounds and compositions can be administered in the form of a dry powder composition or in the form of a liquid spray.

Suppositories for rectal administration can be prepared by mixing one or more compounds or compositions with suitable nonirritating excipients, such as cocoa butter and/or polyethylene glycols, that are solid at room temperature and that melt at body temperature.

For topical administration to the epidermis, the proton pump inhibitors and/or antibacterial compounds can be formulated as ointments, creams or lotions, or as the active ingredient of a transdermal patch. The compounds and compositions can also be administered via iontophoresis. Ointments, creams and lotions can be formulated with an aqueous or oily base with the addition of suitable thickening and/or gelling agents. Alternatively, ointments, creams and lotions can be formulated with an aqueous or oily base and can also contain one or more emulsifying agents, stabilizing agents, dispersing agents, suspending agents, thickening agents, and/or coloring agents. As creams or lotions, the proton pump inhibitors and/or antibacterial compounds can be mixed to form a smooth, homogeneous cream or lotion with, for example, one or more of a preservative (e.g., benzyl alcohol 1% or 2% (wt/wt)), emulsifying wax, glycerin, isopropyl palmitate, lactic acid, purified water, sorbitol solution. Such topically administrate compositions can contain polyethylene glycol 400. To form ointments, the proton pump inhibitors can be mixed with one or more of a preservative (e.g., benzyl alcohol 2% (wt/wt)), petrolatum, emulsifying wax, and Tenox (II) (e.g., butylated hydroxyanisole, propyl gallate, citric acid, propylene glycol). Woven pads or rolls of bandaging material, e.g., gauze, can be impregnated with the transdermally administrable compositions for topical application.

The proton pump inhibitors and/or antibacterial compounds can also be topically applied using a transdermal system, such as one of an acrylic-based polymer adhesive with a resinous crosslinking agent impregnated with the proton pump inhibitors and laminated to an impermeable backing. For example, the proton pump inhibitors and/or antibacterial compounds can be administered in the form of a transdermal patch, such as a sustained-release transdermal patch. Transdermal patches can include any conventional form such as, for example, an adhesive matrix, a polymeric matrix, a reservoir patch, a matrix- or monolithic-type laminated structure, and are generally comprised of one or more backing layers, adhesives, penetration enhancers, and/or rate-controlling membranes. Transdermal patches generally have a release liner which is removed to expose the adhesive/active ingredient(s) prior to application. Transdermal patches are described in, for example, U.S. Pat. Nos. 5,262,165, 5,948,433, 6,010,715 and 6,071,531, the disclosures of which are incorporated by reference herein in their entirety.

The invention provides pharmaceutical kits comprising one or more containers filled with one or more of the ingredients of the pharmaceutical compounds and/or compositions of the invention, including, one or more proton pump inhibitors (e.g., rabeprazole, stereoisomers thereof and/or pharmaceutically acceptable salts thereof) and one or more antibacterial compounds. The proton pump inhibitors and/or antibacterial compounds can be separate components in the kit or can be in the form of a composition in the kit. The kits can also include, for example, other compounds and/or compositions, a device(s) for administering the compounds and/or compositions, and written instructions in a form prescribed by a governmental agency regulating the manufacture, use or sale of pharmaceuticals.

While the proton pump inhibitors of the invention can be administered as the sole active pharmaceutical agent in the methods described herein, they can also be used in combination with one or more compounds which are known to be therapeutically effective against the specific disease that one is targeting for treatment.

Each of the patents and publications cited herein are incorporated by reference herein in their entirety.

It will be apparent to one skilled in the art that various modifications can be made to the invention without departing from the spirit or scope of the appended claims.

1. A method for treating and/or preventing lower esophageal mucosal rings or esophageal strictures in a patient in need thereof by administering a therapeutically effective amount of at least one proton pump inhibitor.

2. The method of claim 1, further comprising, in any order, at least one of dilating the esophagus of the patient; administering an endoscopic examination to the patient; and surgically incising, rupturing and/or excising the lower esophageal mucosal rings or esophageal strictures of the patient.

3. A method for reducing or eliminating a patient's need for dilation of lower esophageal mucosal rings or esophageal strictures by administering to the patient a therapeutically effective amount of at least one proton pump inhibitor.

4. A method for reducing or eliminating a need for surgically incising, rupturing and/or excising lower esophageal mucosal rings or esophageal strictures in a patient by administering a therapeutically effective amount of at least one proton pump inhibitor.

5. The method of any one of claims 1, 3 or 4, wherein the proton pump inhibitor is rabeprazole, omeprazole, esomeprazole, lansoprazole, pantoprazole, a pharmaceutically acceptable salt thereof and/or a stereoisomer thereof.

6. The method of any one of claims 1, 3 or 4, wherein the proton pump inhibitor is a compound of formula (I), a pharmaceutically acceptable salt thereof, and/or a stereoisomer thereof: 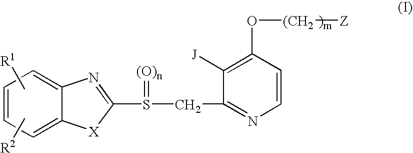
wherein R¹ and R² are each independently a hydrogen atom, a halogen atom, a lower alkyl, lower alkoxy, halogenated lower alkyl, lower alkoxycarbonyl or carboxyl group;
X is —O—, —S—, or ═N—R³, wherein R³ is a hydrogen atom or a lower alkyl, phenyl, benzyl or lower alkoxycarbonyl group; and
Z is:
—(CH₂)ₚ—O—R⁴ 1)
wherein p is an integer for 1 to 3 and R⁴ is hydrogen atom or a lower alkyl, aryl or aralkyl group,
—O—(CH₂)_{q}—R⁵ 2)
wherein q is an integer of 1 to 3 and R⁵ is a halogen atom or an alkoxycarbonyl, aryl or heteroaryl group,
—O—(CH₂)ᵣ—O—(CH₂)ₛ—O—R⁶ 3)
wherein r and s are each independently an integer of 1 to 5 and R⁶ is a hydrogen atom or a lower alkyl group, 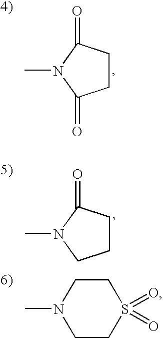
—S(O)ₜ-A 7)
wherein t is an integer of 0 to 2, and A is a lower alkyl, alkoxycarbonylmethyl, pyridyl, furyl 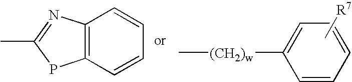
wherein P is —NH—, —O— or —S—, and w is an integer of 0 or 1,
—N(R⁸)—CH₂—C₆H₅ 8)
wherein R⁸ is an acetoxy or lower alkyl group, or
—OR⁹ 9)
wherein R⁹ is a hydrogen atom, a lower alkyl or aryl group;
n is an integer of 0 to 2; m is an integer of 2 to 10, and J is a hydrogen atom or a lower alkyl group, with the proviso that when Z is a group falling under the above category (9), then R⁹ is a lower alkyl group and m stands for an integer of 3 to 10.

7. The method of claim 6, wherein the compound of formula (I) is a compound of formula (A): 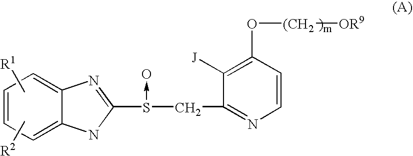
wherein R¹, R², J, m and R⁹ have the same meanings as defined above.

8. The method of claim 6, wherein the compound of formula (I) is a compound of formula (B): 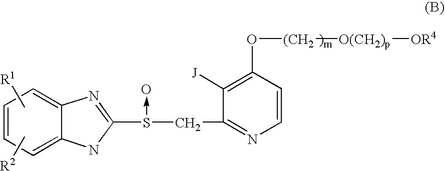
wherein R¹, R², J, p, m and R⁴ have the same meanings as given above.

9. The method of claim 6, wherein the proton pump inhibitor is a compound of formula (C) or a pharmaceutically acceptable salt thereof:

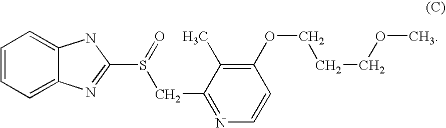

10. The method of claim 6, wherein the proton pump inhibitor is a compound of formula (D) or a pharmaceutically acceptable salt thereof:

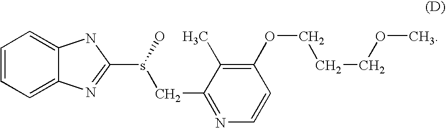

11. The method of claim 6, wherein the proton pump inhibitor is a compound of formula (E) or a pharmaceutically acceptable salt thereof:

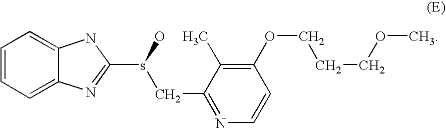

12. The method of claim 5, wherein said proton pump inhibitor is rabeprazole, a pharmaceutically acceptable salt thereof and/or a stereoisomer thereof.

13. The method of claim 12, wherein said proton pump inhibitor is rabeprazole sodium.
